(19) United States
(12) Patent Application Publication
(10) Pub. No.: US 2010/0298797 A1
(43) Pub. Date: Nov. 25, 2010

(54) CONCEALABLE WASTE DISPOSAL WRAP

(76) Inventors: Loranne Ehlenbach, Buffalo Grove, IL (US); John Ehlenbach, Buffalo Grove, IL (US)

Correspondence Address: QUARLES & BRADY LLP, 411 E. WISCONSIN AVENUE, SUITE 2040, MILWAUKEE, WI, 53202-4497, US

(21) Appl. No.: 12/784,124

(22) Filed: May 20, 2010

Related U.S. Application Data
(60) Provisional application No. 61/179,912, filed on May 20, 2009.

(51) Int. Cl. A61F 13/20; B32B 38/10; B32B 7/12

(52) U.S. Cl. 604/385.02; 156/249; 428/343; 428/41.8

(57) ABSTRACT

A wrap is disclosed for containment and concealment of a waste product. The wrap includes a sheet having an inner surface and an outer surface as well as an adhesive that is applied to at least one half of the perimeter of the inner surface of the sheet. When the waste product is placed on the inner surface of the sheet, the adhesive on the inner surface is contactable with another portion of the inner surface to thereby contain the waste product within a sealed volume of the wrap.

### CROSS REFERENCES TO RELATED APPLICATIONS

This application claims priority to U.S. Provisional Patent Application 61/179,912 filed May 20, 2009, which is hereby incorporated by reference as if set forth in its entirety herein.

### BACKGROUND OF THE INVENTION

The invention relates to containers for the inconspicuous transportation and the eventual disposal of personal waste such as tampons.

Almost everyone has been in a situation in which he or she needed to dispose of waste of a personal nature, but was embarrassed to simply place the waste item in a trashcan where someone else may find it. Given the nature of some waste items, it may be desirable to take the waste with you to dispose of it in the privacy of your own home or in such a way that it cannot be identified with you. However, due to the odor, moisture, or appearance of some waste, simply placing the waste item in a pocket or purse would be unreasonable and unsanitary.

Hence there is a need for an improved way to transport and dispose of personal waste.

### BRIEF SUMMARY OF THE INVENTION

A wrap is disclosed for containment and concealment of a waste product. The wrap includes a sheet having an inner surface and an outer surface as well as an adhesive that is applied to at least one half of the perimeter of the inner surface of the sheet. When the waste product is placed on the inner surface of the sheet, the adhesive on the inner surface is contactable with another portion of the inner surface to thereby contain the waste product within a sealed volume of the wrap.

In some forms, the sheet may be made of materials having unique properties or structures. In one form, the sheet may be an expandable film. In another form, folds may be formed within the sheet to allow expansion of the sheet around the waste product when the waste product is deposited in the sheet and the sealed volume formed. Odor absorbing or odor masking materials may also be placed on or in the sheet. A removable shield may cover an exposable face of the odor absorbing or odor masking material. The sheet may be made of a flexible material and may be a single sheet that is foldable onto itself.

In still other forms, the adhesive may cover more than just the perimeter of the inner surface of the sheet. For example, the adhesive may cover one half of the inner surface of the sheet, the entire area of the inner surface of the sheet, or the entire perimeter of the inner surface of the sheet. Regardless of the pattern of the adhesive, a removable adhesive shield may cover an exposable face of the adhesive.

In some forms, the outer surface of the sheet may have visual markings formed thereon for disguising the nature of the contents of the wrap.

A wrap kit is also disclosed including a wrap of the kind described above and further including a flexible, disposable pouch which encases the wrap prior to use.

In some forms, the wrap kit may further include a clean tampon or a sanitary disposable glove contained within the flexible, disposable pouch.

A related method of disposing of a waste product using the wrap is also disclosed. A wrap of the type described above is provided which includes a removable adhesive shield that covers an exposable face of the adhesive on the inner surface of the sheet. The adhesive shield is removed from the adhesive. A waste product is placed on the inner surface of the wrap. Then, the inner surface of the wrap is folded onto itself thereby covering the waste product. A pressure is applied along the perimeter of the wrap to activate the adhesive to seal the wrap and thereby contain a waste product in a formed sealed volume.

In some forms, the method may further include providing a tampon within the wrap to replace a used tampon.

In other forms, the method may further include providing a sanitary glove with the wrap to allow for sanitary handling of the waste product with the wrap. In this form, the waste product ultimately placed on the inner surface may be the sanitary glove.

These and still other advantages of the invention will be apparent from the detailed description and drawings. What follows is merely a description of preferred embodiments of the present invention. To assess the full scope of the invention the claims should be looked to as the preferred embodiments are not intended to be the only embodiments within the scope of the claims.

### BRIEF DESCRIPTION OF THE DRAWINGS

FIG. 1 is a top view showing the inside of a wrap;

FIG. 2 is a bottom view showing the outside of the wrap of FIG. 1;

FIG. 3 is a side view taken through line 3-3 of FIG. 1;

FIG. 4 is a side view of an alternative embodiment of a wrap construction in which the wrap further includes an odor masking or odor absorbing layer;

FIG. 5 is a top view of a pouch for packaging a wrap and another article for packaging with the wrap;

FIG. 6 is a top view of an alternative form of a wrap including integral folds that permit the wrap to expand to better accommodate a waste product; and

FIG. 7 is a top view of the inner surface of an embodiment of a wrap made of an expanding film.

### DETAILED DESCRIPTION OF THE INVENTION

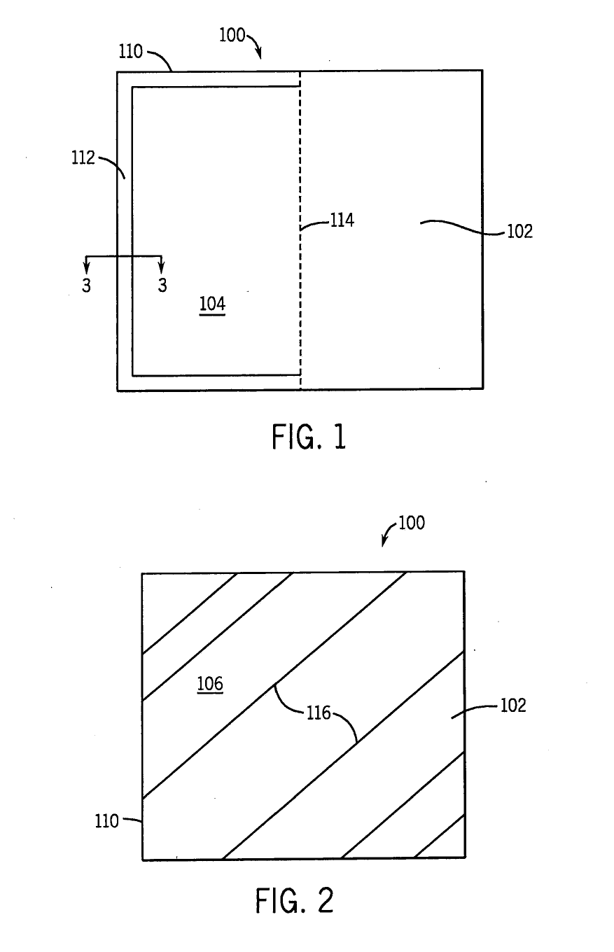

Referring first to FIGS. 1 through 3, a generally rectangular-shaped wrap 100 is shown for the containment and concealment of a waste product or item of a personal nature. The wrap 100 includes a sheet 102 of a moisture-proof and flexible material with an inner surface 104 and an outer surface 106 on the opposing faces of the sheet 102. Materials appropriate for fabrication of the sheet 102 include, but are not limited to, coated paper and plastic as well as the sheet materials which are described in more detail below. Moisture-proofing can be achieved by selecting a material for the sheet 102 that is moisture proof or by treating the sheet 102 with a moisture-proof coating or the like. The wrap 100 may be made of a single material or multiple materials formed or bonded together into a single sheet to achieve these properties.

In the form of the wrap 100 shown in FIGS. 1 through 3, an adhesive 108 is located on an area on the inner surface 104 around one-half of a perimeter 110 of the sheet 102. Prior to activation of the adhesive 108, the adhesive 108 is covered by an adhesive shield 112 on the side of the adhesive 108 opposite the inner surface 104. In other forms, an adhesive may cover different areas of the sheet 102 such as, for example, the entire perimeter 110 of the inner surface 104, one half of the inner surface 104, or even the entire inner surface 104. Upon activation (i.e., removal of the adhesive shield 112 and attaching the exposed face of the adhesive 108 to another portion of the inner surface 104 as will described in more detail below), the adhesive 108 is suitable to form a moisture-proof seal such that any moisture or odor associated with the moisture of the waste product to be contained in the wrap 100 is not easily transported through the adhesive 108 or the seal formed by the adhesive 108. It is contemplated that if the adhesive 108 is moisture-proof and covers the entirety of the inner surface 104 of the sheet 102, that the adhesive 108 itself may act as a coating on the sheet 102 which would render the wrap 100 moisture-proof.

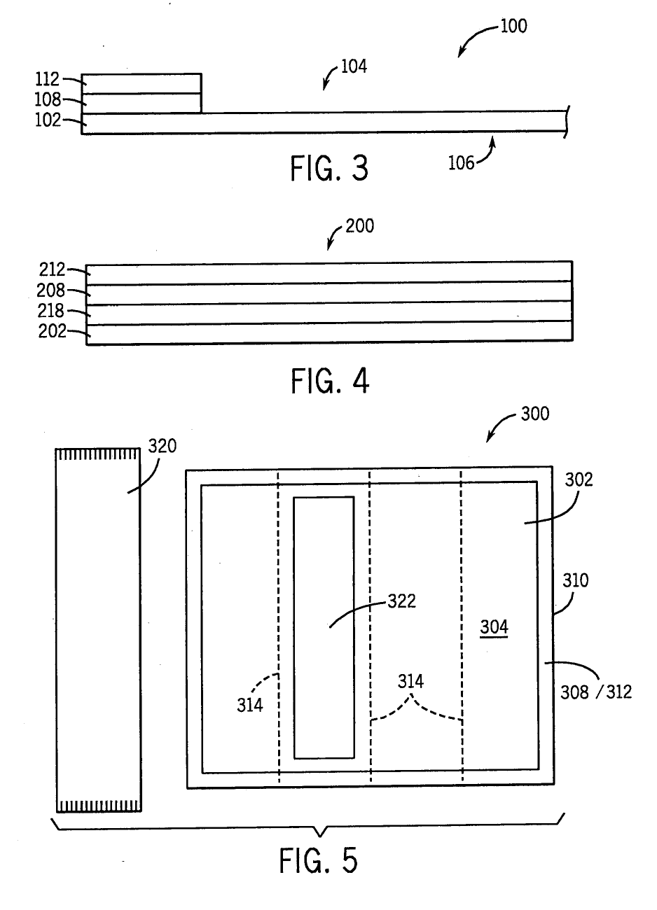

As best illustrated in FIG. 3, the removable adhesive shield 112 is placed over the adhesive 108, such that the wrap 100 can be folded or rolled into a smaller size for storage or carrying prior to use without the adhesive 108 bonding the contacting inner surfaces 104 of the wrap 100 together. Typically, the adhesive shield 112 is a film of disposable material to which the adhesive 108 is unable to form a strong bond. This adhesive shield 112 is then selectively removable under a weak pulling force to expose the side of the adhesive 108 that is not already bonded to the inner surface 104.

To dispose of a waste product, such as a used tampon, in the wrap 100, the following steps may be taken. First, the waste item is placed on the inner surface 104 of the sheet 102, and the adhesive shield 112 is removed to expose the adhesive 108. Then, the sheet 102 is folded over onto itself along a fold line 114 to bring the two sides (one including the adhesive perimeter and the other not including the adhesive perimeter) of the inner surface 104 into contact with one another. As the adhesive 108 around one half of the perimeter 110 is exposed, the application of pressure over the adhesive-covered areas will form a complete moisture-proof seal around the portion of the newly formed perimeter. In conjunction with the seal existing along the fold line 114, this effectively captures and contains the personal waste product within a sealed volume of the wrap 100 such that the sheet 102 contains the waste and prevents any leakage or wicking of the contents from the sealed volume. The contained waste product can now be transported or disposed of as desired by the user within the sealed wrap.

The flexible nature of the sheet 102 will allow the wrap 100 to contain a waste product without occupying much excess volume, unlike a container composed of a more rigid material such a cardboard or fiberboard. By making a wrap 100 out of non-rigid and flexible material, the wrap 100 may have a flexible form factor that makes it easier to carry in a purse, bag, or pocket, either with or without a waste product.

The outer surface 106 may also incorporate visual markings 116 as illustrated in FIG. 2 or be opaque to hamper the visual identification of the contents within the wrap 100. The visual markings 116 may be visual designs, patterns, pictures or other visual representations, and further disguise the contents and purpose of the wrap 100. The visual markings 116 may also enhance the aesthetic appeal of the outer surface 106 or may include words such as brand names or logos.

Turning now to FIG. 4, a side view of an alternative embodiment of a wrap 200 is shown. In this form, the wrap 200 includes a sheet 202 which includes an additional intermediate layer 218 of odor masking or absorbing material, which may be placed on or in the sheet 202. An odor masking material may be one that emits a pleasant scent, such that the scent covers or masks the odor of the contents of the wrap 200. An odor absorbing material may be one that has odor neutralizing properties and reduces or eliminates the odor of the contents of the wrap 200. In any event, the odor masking or absorbing material helps to reduce the identification of the contents of the wrap 200 by odor. In addition, reducing or masking the odor of the contents of the wrap 200 allows the wrap 200 to be transported or stored for longer periods of time to allow for the disposal of the waste at a later time and place.

In the form illustrated in FIG. 4, the adhesive layer 208 is deposited upon the odor masking layer 218 and a releasable adhesive shield 212 initially covers the adhesive layer 208. It is contemplated that the coverage of the odor masking or absorbing materials by the adhesive shield 212 may prevent pre-mature degradation of the odor absorbing or emitting (as in the case of masking) qualities of the layer 218.

Referring now to FIG. 5, a disposable, flexible pouch 320 is illustrated in which a wrap 300 and/or an included article 322, such as a tampon and/or a glove, may be packaged. The flexible pouch 320 assists in keeping the wrap 300 and included article 322 together and further may assist in preventing damage to the wrap 300 prior to use. The disposable, flexible pouch 320 is made of a material of such thickness and strength that the wrap 300 will be protected from punctures and abrasions when carried in a pocket, purse, or similar location. Materials appropriate for the pouch 320 include, but are not limited to, coated paper and plastic of an appropriate durability to protect the wrap 300. The ends of the pouch 320 may be sealed to ensure the integrity of the pouch 320 and the items contained therein.

It is noted that the wrap 300 in FIG. 5 differs from those shown in FIGS. 1 through 4 in that the wrap 300 has include an adhesive 308 and a releasable adhesive shield 312 located about the entire periphery 310 of the inner surface 304 of the sheet 302. By include adhesive 308 about the entire periphery 310, may reduce the likelihood of a gap forming during sealing which allows moisture to leak from the sealed volume and further accommodates folding in more than one direction. Further, to fit in the pouch 320, the wrap 300 in FIG. 5 is indicated as having multiple fold lines 314.

To insert the wrap 300 and article 322 into the pouch, the wrap 300 may be folded or rolled around the included article 322 to form a wrap package. Alternatively, the wrap 300 and/or any included article 322 may be folded separately in order to minimize the total size of the items within the pouch 320.

The user opens the pouch 320 to provide access to the wrap 300 and the included article 322. Once opened, the wrap 300 may be used to dispose of personal waste as described above and, further, the wrap 300 may be used to contain additional waste such as the now-opened pouch 320 and/or any other waste, such as a used glove if the glove is the included article 322.

In some configurations, this pouch 320 may be separate from a package containing a tampon. In other configurations, the pouch 320 and the packaging of the tampon may be adhered or otherwise coupled to one another. If the wrap 300 has been encased in the flexible pouch 320 before use, then it may less likely to be damaged than if it is included in packaging that also houses a new tampon.

A wrap of the kind described herein is a single-use disposal wrap and is not a repurposed item of packing for disposal. Such re-purposed packaging is likely to have holes, tears, and other abrasions as the result of damage from opening the packaging or being transported with other items in a purse, pocket, or the like. Further, to the extent adhesive might be used in such packaging, the adhesive would be de-adhered to remove a new product and the re-adhered to dispose of the used product, thus compromising the quality of the adhesive seal. As previously discussed, if the wrap was hindered in such a way, it would no longer serve its purpose of allowing the user to transport the waste to a more convenient location in a sanitary manner, or to dispose of the waste in a trashcan without leaving a potentially odorous, unsanitary, and embarrassing item.

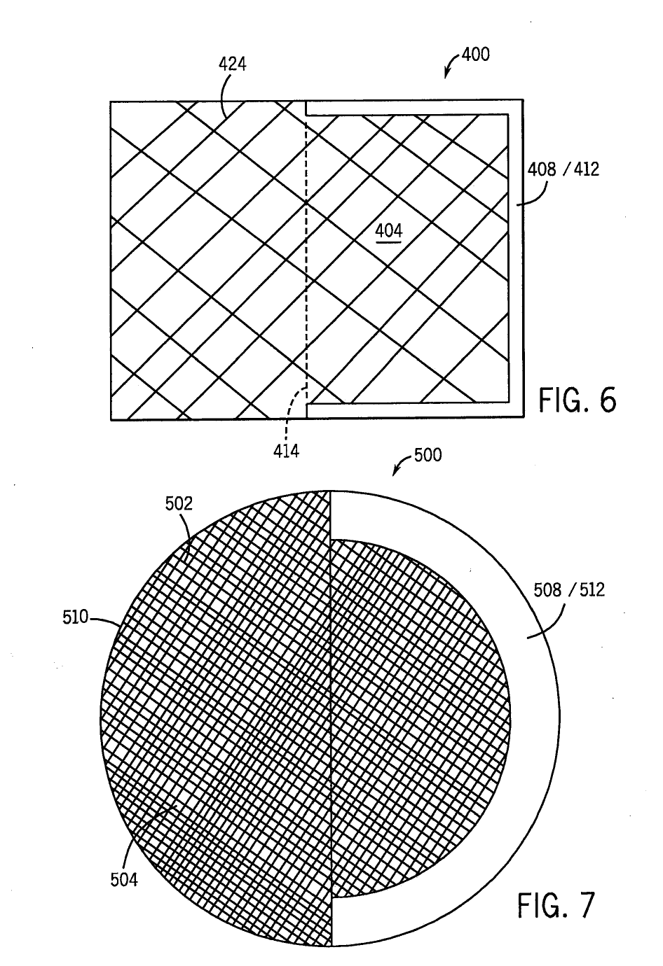

Expanding folds can be used to allow otherwise flat sheets of material to expand. In wrap 400 of FIG. 6, the expanding folds 424 on an inner surface 404 allow the wrap 400 to better accommodate larger amounts of waste or waste having a significant third dimension.

Since the exact size and nature of waste cannot always be predicted, the materials out of which the wrap is made may incorporate expanding films in the material of the sheet. Referring now to FIG. 7, a circular wrap 500 shows such an embodiment incorporating an expanding film in the sheet 502. Examples of expanding films include, but are not limited to, structural elastic-like films, elastomeric films, or elastic laminates. By using an expanding film, the wrap 500 can better accommodate larger amounts of waste or waste that may possibly puncture less elastic materials. Expanding films allow the inner surface 504 to stretch and distribute forces. This allows for expansion for larger amounts of waste and resistance to punctures caused by excessive force on a focused point load on the inner surface 504.

Moreover, FIG. 7 illustrates that the wrap 500 may have a non-rectangular form. As the wrap 500 is circular, a portion of the inner surface 504 following the circumference 510 includes the adhesive 508 and the adhesive shield 512.

Hence, as the disclosed wraps provides a full adhesive seal around the personal waste, the wraps disguise the appearance of the personal waste, absorb and/or mask the odor of the personal waste, and contain any moisture associated with the personal waste. As such, once the waste is contained within a wrap, the personal waste is better suited for transport to a more convenient disposal location or can be placed in a trashcan without offending the owner of the facility or embarrassing the user. Without this full adhesive seal, transportation or disposal of the waste may be unreasonable or unsanitary. Therefore, the disclosed wrap is better suited than a bag that uses tape, ties, and/or snaps to dispose of waste of a personal nature.

Many modifications and variations to this preferred embodiment will be apparent to those skilled in the art, which will be within the spirit and scope of the invention. Therefore, the invention should not be limited to the described embodiment. To ascertain the full scope of the invention, the following claims should be referenced.

What is claimed is:

1. A wrap for containment and concealment of a waste product, the wrap comprising:
a sheet having an inner surface and an outer surface; and
an adhesive applied to at least one half of a perimeter of the inner surface of the sheet;
wherein, when the waste product is placed on the inner surface of the sheet, the adhesive on the inner surface is contactable with another portion of the inner surface to thereby contain the waste product within a sealed volume of the wrap.

2. The wrap of claim 1, wherein the sheet comprises an expandable film.

3. The wrap of claim 1, wherein folds are formed within the sheet to allow expansion of the sheet around the waste product when the waste product is deposited in the sheet and the sealed volume formed.

4. The wrap of claim 1, wherein odor absorbing materials are placed on or in the sheet.

5. The wrap of claim 4, further comprising a removable shield that covers an exposable face of the odor absorbing materials.

6. The wrap of claim 1, wherein odor masking materials are placed on or in the sheet.

7. The wrap of claim 6, further comprising a removable shield that covers an exposable face of the odor masking materials.

8. The wrap of claim 1, wherein the adhesive is applied to one half of the inner surface of the sheet.

9. The wrap of claim 1, wherein the adhesive is applied to an entire area of the inner surface of the sheet.

10. The wrap of claim 1, wherein the outer surface of the sheet has visual markings formed thereon.

11. The wrap of claim 1, further comprising a removable adhesive shield that covers an exposable face of the adhesive applied to the inner surface of the sheet.

12. The wrap of claim 1, wherein the sheet is made of a flexible material.

13. The wrap of claim 1, wherein the sheet is a single sheet.

14. A wrap kit comprising:
a wrap including a sheet having an inner surface and an outer surface and an adhesive applied to at least one half of a perimeter of the inner surface of the sheet wherein, when a waste product is placed on the inner surface of the sheet, the adhesive on the inner surface is contactable with another portion of the inner surface to thereby contain the waste product within a sealed volume of the wrap; and
a flexible, disposable pouch which encases the wrap prior to use.

15. The wrap kit of claim 14, further comprising a clean tampon contained within the flexible, disposable pouch.

16. The wrap kit of claim 14, further comprising a sanitary disposable glove contained within the flexible, disposable pouch.

17. A method of disposing of a waste product, the method comprising:
providing a wrap comprising a sheet having an inner surface and an outer surface and an adhesive applied to at least one half of a perimeter of the inner surface of the sheet, and a removable adhesive shield that covers an exposable face of the adhesive on the inner surface of the sheet;
removing the adhesive shield from the adhesive;
placing a waste product on the inner surface;
folding the inner surface onto itself thereby covering the waste product; and
applying pressure along the perimeter of the wrap to activate the adhesive to seal the wrap and thereby contain a waste product in a sealed volume.

18. The method of claim 17, further comprising the step of providing a tampon within the wrap to replace a used tampon.

19. The method of claim 17, further comprising the step of providing a sanitary glove with the wrap to allow for sanitary handling of the waste product with the wrap.

20. The method of claim 19, wherein the step of placing the waste product on the inner surface includes placing the sanitary glove on the inner surface.
